(19) United States
(12) Patent Application Publication
(10) Pub. No.: US 2026/0263827 A1
(43) Pub. Date: Sep. 10, 2026

(54) DEVICE FOR TRANSMITTING SOUND FREQUENCIES THROUGH LIGHT USING INFRARED AND LED EMITTERS FOR HEALTH AND WELL-BEING APPLICATIONS

(71) Applicants: Erika Macedo Campante Fittipaldi Torga, Parkland, FL (US); Alfredo Pereira Afonso, Almirante Tamandare (BR)

(72) Inventors: Erika Macedo Campante Fittipaldi Torga, Parkland, FL (US); Alfredo Pereira Afonso, Almirante Tamandare (BR)

(21) Appl. No.: 19/457,978

(22) Filed: Jan. 23, 2026

(30) Foreign Application Priority Data
Feb. 2, 2025 (BR) BR1020250033984

(51) Int. Cl. A61N 5/06

(52) U.S. Cl. CPC A61N 5/0613; A61N 2005/0626; A61N 2005/0645; A61N 2005/0651; A61N 2005/0659

(57) ABSTRACT

An electronic device is disclosed for transmitting sound frequency information through modulated light emissions using infrared and light-emitting diode (LED) emitters. The device receives sound frequency signals via a wired or wireless input interface and converts the signals into electrical modulation signals that control one or more light emitters to emit infrared and LED light modulated according to the characteristics of the sound frequency signals. The emitted light functions as an active light-based emission carrying sound frequency information for interaction with biological systems. The device may be implemented in multiple physical configurations, including wearable units, animal-mounted devices, handheld emitters, and projection-based systems. The disclosed system enables non-invasive interaction with biological systems for health, well-being, and biological interaction applications without tissue penetration or implantation.

### TECHNICAL FIELD

The present disclosure relates to electronic devices configured to transmit sound frequencies through modulated light emissions. More specifically, the disclosure relates to systems intended for health, well-being, and biological interaction applications, including non-invasive interaction with human, animal, and plant systems through infrared and light-emitting diode (LED) based light modulation carrying sound frequency information.

### BACKGROUND

Sound frequencies have been studied and applied in various contexts related to biological interaction, relaxation, stimulation, and well-being. Separately, light-based systems utilizing infrared and LED emitters have been employed in multiple non-invasive applications involving interaction with biological tissues.

Existing solutions typically rely on light emissions alone or sound emissions alone as the primary active modality for biological interaction. In many cases, sound-based systems transmit acoustic signals directly through air or physical media, while light-based systems emit infrared or LED light without dynamic modulation based on sound frequency information. As a result, such systems do not provide an efficient or integrated mechanism by which sound frequency information is encoded into active light emissions in a controlled and synchronized manner

Accordingly, there exists a need for a compact, scalable, and low-cost electronic device capable of transmitting sound frequency information through light emitters for health and well-being related applications, without invasive procedures.

### SUMMARY OF THE DISCLOSURE

The present disclosure provides an electronic device configured to receive sound frequency signals and convert such signals into modulated light emissions using infrared and LED emitters.

In one aspect, the device comprises an electronic circuit including a power source, a signal input interface, modulation components, and one or more light emitters configured to emit pulsed light corresponding to received sound frequency signals.

In certain applications, the disclosed device may be used in health and general well-being contexts, where sound frequency information is transmitted through light to interact with biological systems in a non-invasive manner. The device is not intended to diagnose or cure specific medical conditions, but rather to provide a technological platform for sound-frequency-based interaction applicable to health support, relaxation, biological stimulation, and exploratory or complementary use.

The device may be implemented in multiple physical configurations, including wearable devices, animal collars, projection units, and handheld emitters.

### BRIEF DESCRIPTION OF THE DRAWINGS

FIG. 1 illustrates an electronic circuit diagram of the sound frequency to light modulation system.

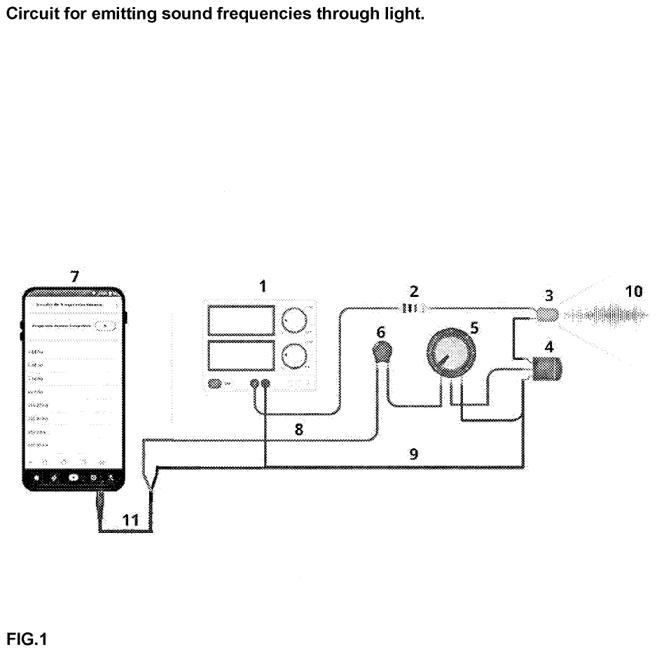

FIG. 2 illustrates a wearable embodiment configured for attachment to a human wrist.

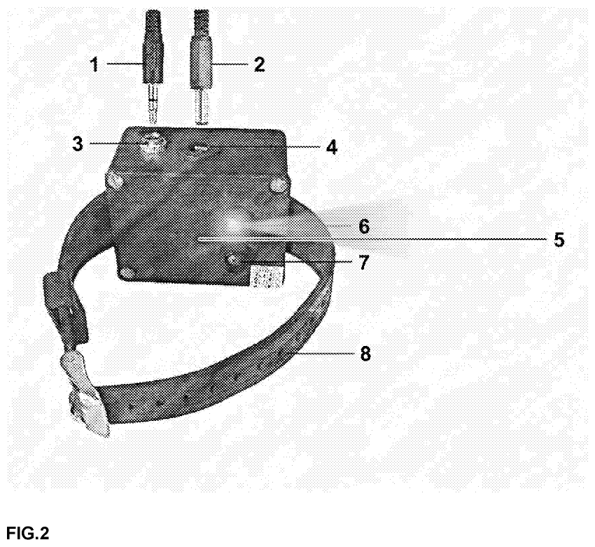

FIG. 3 illustrates a wearable embodiment including a display and wireless connectivity.

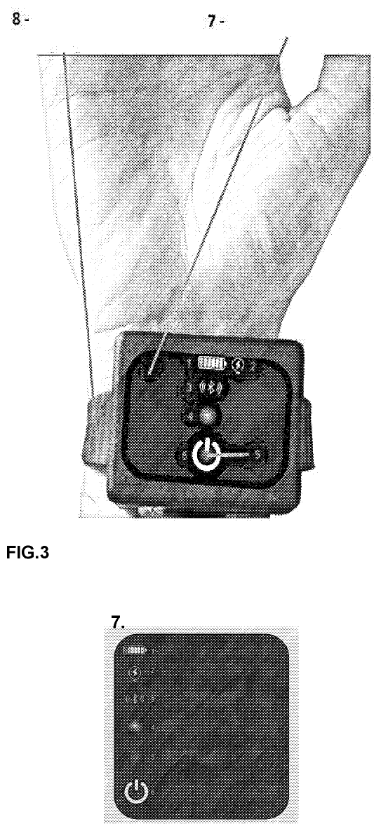

FIG. 4 illustrates an embodiment configured for use with animals.

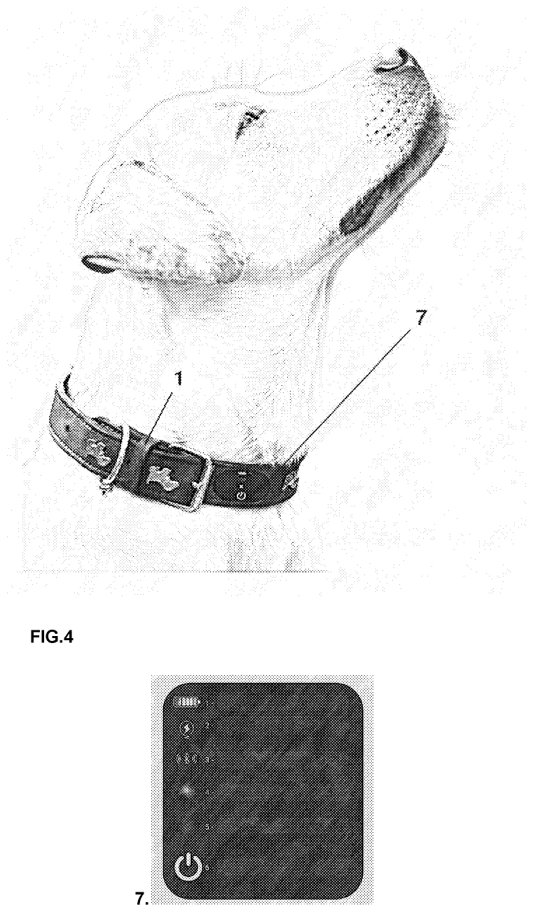

FIG. 5 illustrates an internal view of an animal collar embodiment.

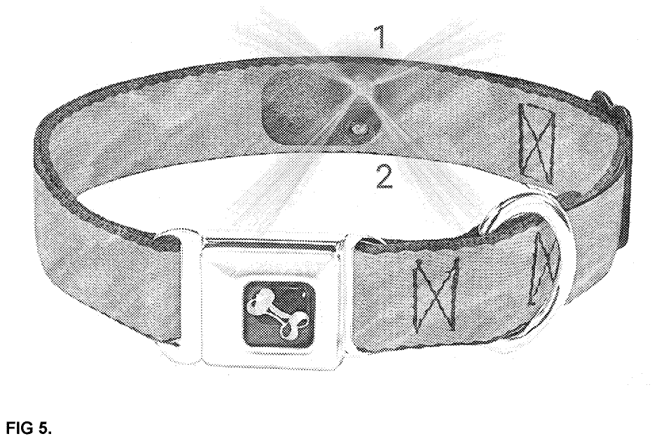

FIG. 6 illustrates a projection-based embodiment for interaction with plants.

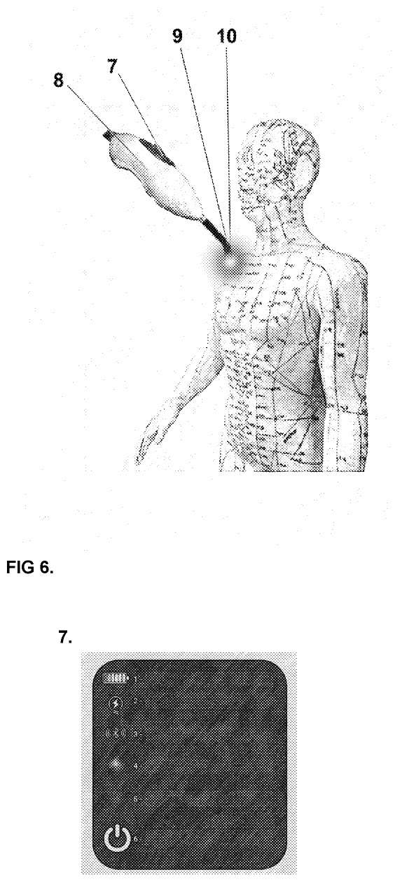

FIG. 7 illustrates a handheld projection embodiment for directed application.

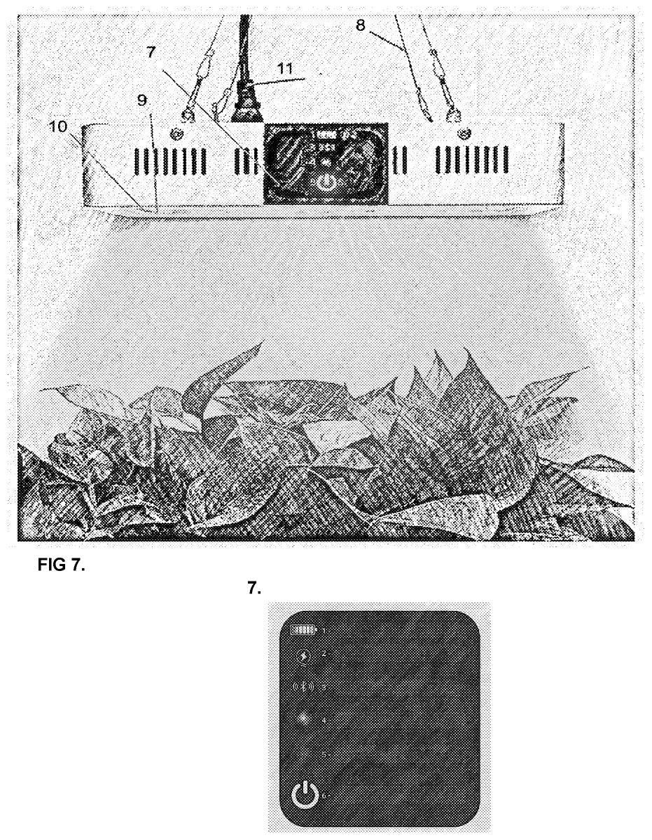

### DETAILED DESCRIPTION

### General Architecture

The disclosed device comprises a compact electronic circuit configured to receive sound frequency signals via wired or wireless input and to modulate one or more light emitters accordingly.

The electronic circuit may include, but is not limited to:
a power supply, including a battery or external DC source;
a signal input interface, such as an audio connector or wireless communication module;
signal modulation components including transistors, resistors, capacitors, potentiometers, and related electronic elements;
one or more light emitters, including infrared, near-infrared, and LED emitters.

The circuit converts incoming sound frequency signals into electrical modulation signals that control the emission pattern of the light emitters, causing the emitters to pulse in accordance with the characteristics of the sound frequency.

### Wearable Human Embodiment

In one embodiment, the device is configured as a wearable unit adapted for placement on a human body, such as on the wrist.

The wearable housing contains the electronic circuit and includes one or more light emitters positioned to face the skin. The device may include visual indicators for power status, signal transmission, battery level, and wireless connectivity.

This embodiment allows non-invasive interaction between modulated sound frequencies and the human body for health and well-being support.

### Animal Embodiment

In another embodiment, the device is configured for attachment to animals using a collar or similar fastening mechanism.

The housing may be reinforced for durability and includes light emitters positioned to face the animal's body. Wireless signal transmission may be used to facilitate operation without physical connections.

This embodiment allows non-invasive interaction with animals for well-being and biological interaction purposes.

### Plant Projection Embodiment

In another embodiment, the device is configured as a projection unit capable of emitting modulated light toward plants from a distance.

The projection unit may include directional elements such as reflectors, diffusers, or lenses to control the emission area, allowing interaction with plants for biological stimulation and health-related growth support.

### Handheld Projection Embodiment

In another embodiment, the device is configured as a handheld projection unit that allows a user or professional to manually direct modulated light emissions toward selected target areas on humans, animals, or plants.

### Signal Modulation and Transmission

Sound frequency signals received by the device are converted into electrical modulation signals that control the operation of the light emitters, causing the emitters to pulse according to the sound frequency characteristics.

Modulation techniques may include analog modulation, digital modulation, pulse-width modulation, or combinations thereof.

### Method of Use

In use, a user or professional selects a desired sound frequency signal and activates the device. The sound frequency signal is transmitted to the electronic circuit via a wired or wireless input interface.

The electronic circuit converts the sound frequency signal into electrical modulation signals that control the operation of one or more light emitters, causing the emitters to pulse according to the sound frequency characteristics.

The device may be operated in continuous or intermittent modes and may be used in self-administered or professionally assisted applications.

### Variations and Alternative Embodiments

The disclosed system is not limited to the embodiments described herein.

Variations may include:
different types of light emitters across infrared, near-infrared, and visible spectra;
multiple emitters operating simultaneously;
different sound frequency sources and signal formats;
integration into existing electronic systems or wearable platforms;
applications related to health support, well-being, relaxation, research, agriculture, and biological exploration.

The disclosed system operates in a non-invasive manner, without penetration of tissue or internal implantation, relying solely on external application of modulated light emissions for interaction with biological systems.

The disclosed embodiments may be used in professional environments, including clinical, veterinary, agricultural, or research settings, as well as in domestic or personal use environments by end users.

### CONCLUSION

The present disclosure provides a versatile electronic system for transmitting sound frequencies through modulated light emissions in a non-invasive manner, suitable for health, well-being, and biological interaction applications across multiple physical configurations.

What is claimed is:

1. An electronic device comprising:
a power source;
a signal input interface configured to receive sound frequency signals;
a modulation circuit configured to convert the sound frequency signals into electrical modulation signals; and
one or more light emitters including infrared emitters, light-emitting diode (LED) emitters, or combinations thereof,
wherein the modulation circuit controls the one or more light emitters to emit light modulated according to characteristics of the sound frequency signals, and
wherein the emitted light functions as an active light-based emission carrying sound frequency information for interaction with biological systems.

2. The electronic device of claim 1, wherein the signal input interface comprises a wired audio input, a wireless communication interface, or a combination thereof.

3. The electronic device of claim 1, wherein the modulation circuit is configured to perform analog modulation, digital modulation, pulse-width modulation, or combinations thereof.

4. The electronic device of claim 1, wherein the device is configured as a wearable unit adapted for placement on a human body.

5. The electronic device of claim 1, wherein the device is configured for attachment to an animal using a collar or fastening mechanism.

6. The electronic device of claim 1, wherein the device is configured as a projection-based unit that emits the modulated light toward plants or other biological targets from a distance.

7. The electronic device of claim 1, wherein the device is configured as a handheld unit that allows a user to manually direct the modulated light toward a selected target area.

8. The electronic device of claim 1, further comprising a control interface configured to adjust one or more parameters of the sound frequency signals or the light emission.

9. The electronic device of claim 1, wherein the emitted light is configured to operate in continuous or intermittent emission modes.

10. The electronic device of claim 1, wherein the device is configured for non-invasive interaction with biological systems without tissue penetration or implantation.
